(19) United States
(12) Patent Application Publication
(10) Pub. No.: US 2021/0239672 A1
(43) Pub. Date: Aug. 5, 2021

(54) Method for Evaluation of Similarity between Dietary Fats to Human Milk Fat

(71) Applicant: JIANGNAN UNIVERSITY, Wuxi, Jiangsu (CN)

(72) Inventors: Xingguo WANG, Wuxi, Jiangsu (CN); Wei WEI, Wuxi, Jiangsu (CN); Qingzhe JIN, Wuxi, Jiangsu (CN)

(21) Appl. No.: 16/956,585

(22) PCT Filed: Dec. 17, 2018
(86) PCT No.: PCT/CN2018/121493

§ 371 Date: Jun. 20, 2020

(30) Foreign Application Priority Data
Dec. 21, 2017 (CN) 201711392850.0

(51) Int. Cl. G01N 33/06; A23L 33/00

(52) U.S. Cl. CPC G01N 33/06; A23V 2002/00; A23L 33/40

(57) ABSTRACT

This invention discloses a method for similarity evaluation of dietary fat, including, establishing the evaluation model and quantifying the differences between dietary fat and human milk fat. The evaluation model provided by the present invention can comprehensive highlight of similarity without the influence of the content of indexes, integrated quantitative differences between dietary fat and human milk fat using similarity coefficient. The similarity evaluation method can grade human milk fat objectively and evaluate the similarity of dietary fat to human milk fat through the grading evaluation. Thus the invention provides a comprehensive method to evaluate the similarity of dietary fat to human milk fat.

### BACKGROUND OF THE INVENTION

### 1. Technical Field

This invention generally relates to infant formula food products field, and more specifically to a method for determining the similarity of the fat of infant formula food products to human milk fat.

### 2. Background Art

Human milk is commonly considered as the best food for infants because it contains numerous functional constituents for infants' growth and development.

Throughout the development of infant formula, it is a process history for mimicking human milk, especially with respect to fat. The fat used in infant formula food belongs to human milk fat substitutes. It was initially produced according to the ratio of fat in human milk. then the essential fatty acids and functional long-chain polyunsaturated fatty acids such as docosahexaenoic acid (DHA), arachidonic acid (AA), were added to infant formulas based on the fatty acid composition and distribution of human milk fat. The ultimate aim of human milk fat substitutes is to mimic the chemical structure including fatty acids, triacylglycerols and complicated lipids composition, and physical properties including milk fat globule and sensory characteristics of human milk fat as close as possible.

So far there is no method for evaluating human milk fat substitute in which the lipid composition of human milk fat is thoroughly considered. For example, no method is available presently for the nutrients that are important for the intellectual development of infants but with very low content in human milk fat. Therefore, we need an objective, scientific and comprehensive evaluation method for quantifying the similarity between human milk fat substitute and human milk fat. Meanwhile, it is necessary to establish a comprehensive evaluation model to consider all aspects of the lipid composition of human milk fat and the whole similarity of human milk fat substitute.

### SUMMARY OF THE INVENTION

The present invention provides an objective, scientific and comprehensive method for similarity evaluation of dietary fat, and briefly introduces some better embodiments. Perhaps it should be noted that some simplifications or omissions may be made in the present context to make it clear, but such simplifications or omissions may not be used to limit the scope of the present invention.

In view of the problems in the above and/or existing evaluation models for dietary fat, the present invention is proposed.

The purpose of the present invention is to fill in the gaps in the prior art and to provide an evaluation method that takes into account all aspects of lipid compositions comprehensively.

The present invention provides improved procedures for evaluating the similarity of dietary fat to human milk fat. The actual values of a certain index in dietary fat are comparing with the range of standard values of the corresponding index in human milk fat to calculate the drift rate of each index of dietary fat by the use of the equation:

$$C = \frac{\vert b - a \vert}{a}$$

in which, a represents the standard value of an index, b represents the actual value of the index. If b is more than amax, a is equivalent to amax; and if b is less than amin, a is equivalent to amin. C, represents the extent of the determined value of a certain index deviating from the theoretical value range, i.e. shift rate; When b is between amin and amax, C is defined as 0, namely, shift rate is equivalent to 0.

a similarity evaluation model is established using the equation:

$$G = {\sum }_{i = 1}^{n} [ ( 1 - \frac{\vert b - a \vert}{a} ) \times 100 ] \times \frac{{d}_{i}}{{\sum }_{i = 1}^{n} {d}_{i}}$$

where dᵢ represents the appointed weight for i^{th} index; d₁/Σᵢ₌₁ⁿdᵢ represents the normalized weight for the i^{th} index after normalization; G represents the similarity coefficient of a certain index of dietary fat; the higher G value, the higher the similarity; and the maximum of G value is 100; conversely, the smaller G value, the lower similarity.

What is claimed is that it comprises conducting a hierarchical evaluation of multi-grade indexes of dietary fat to obtain the similarity through the model as follow:

$${G}_{III} = {\sum }_{i = 1}^{n} [ ( 1 - \frac{\vert b - a \vert}{a} ) \times 100 ] \times \frac{{d}_{i}}{{\sum }_{i = 1}^{n} {d}_{i}}$$

$${G}_{II} = {\sum }_{i = 1}^{n} {e}_{i} {G}_{III}$$

$${G}_{I} = {\sum }_{i = 1}^{n} {f}_{i} {G}_{II}$$

in which, G_{III} represents the similarity coefficient of each grade III index of dietary fat to human milk fat; G_{II} represents the similarity coefficient of each grade II index of dietary fat to human milk fat; e represents the appointed weight for the i^{th} grade III index, 0≤eᵢ≤1; G_{I} represents the similarity coefficient of all grade II index of dietary fat to human milk fat; fᵢ represents the appointed weight for the i^{th} grade II index, 0≤fᵢ≤1.

The standard value of a certain index, a, is expressed as mean±n times of standard deviation (a=ā±nσ). Preferably, a is the 95% confidence interval, n=1.96.

With respect to the application of the method of this patent disclosure to dietary fat, it should be specifically noted that the grade I index is the lipid composition of human milk fat.

The grade II index contains fatty acids, sn-2 fatty acids, triacylglycerols and complicated lipids.

The fatty acids in grade III index contain major fatty acids, trace fatty acids and special fatty acids; The sn-2 fatty acids in grade III index comprises major sn-2 fatty acids, trace sn-2 fatty acids and special sn-2 fatty acids.

The triacylglycerols in grade III index contain major triacylglycerols and trace triacylglycerols.

The complicated lipids in grade III index contain phospholipids, sterols, and glycolipids.

The major fatty acids are the fatty acid that represents more than 1% of the total fatty acids; major sn-2 fatty acids are the fatty acids that represent more than 1% of the total sn-2 fatty acids; major triacylglycerols are the triacylglycerols that represent more than 1% of the total triacylglycerols; trace fatty acids are defined as the fatty acids with less than 1% of the total fatty acids, except for special fatty acids; trace sn-2 fatty acids are defined as the fatty acids with less than 1% of the total sn-2 fatty acids, except for special fatty acids; trace triacylglycerols are defined as the triacylglycerols with less than 1% of the triacylglycerols; special fatty acids that are different from conventional fatty acids due to their special structure of functional group contain odd-chain fatty acids, branched-chain fatty acids, cyclic fatty acids, alkenyl acid.

### BRIEF DESCRIPTION OF THE DRAWINGS

In order to illustrate the of this invention, the preparation of the appended drawings used in the below embodiments is described. Obviously, those skilled in the art will appreciate that other drawings may be obtained according to these without creative labour.

FIG. 1 depicts the content distribution of a certain index in human milk fat. When the sample size is adequately large, the content appears in the normal distribution.

FIG. 2 shows the schematic of the hierarchical evaluation system for the similarity of multistage indexes between dietary fat and human milk fat.

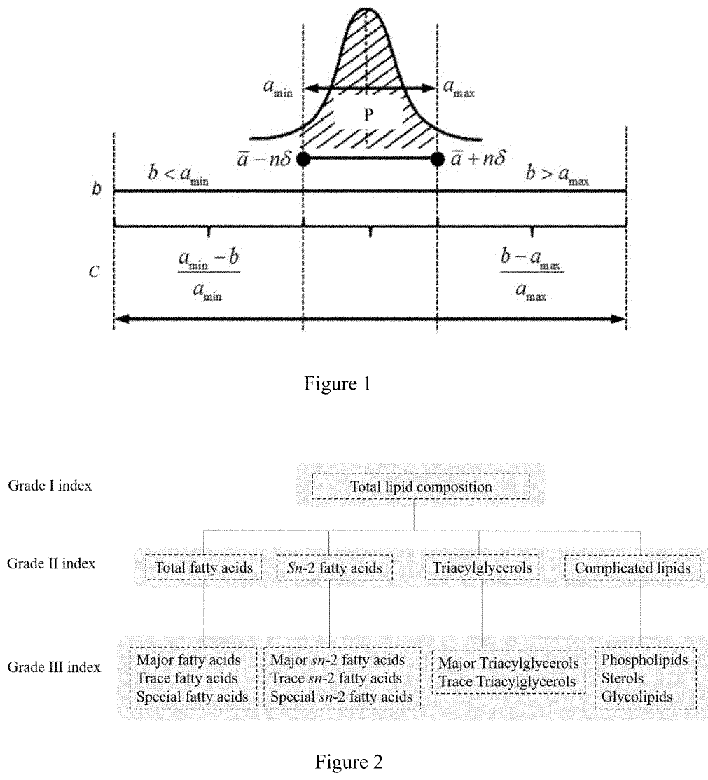

### DESCRIPTION OF PREFERRED EMBODIMENTS

The following examples serve to illustrate the application of this invention in combination with the appended drawings of the specification. It is obvious that the preferred embodiments of this invention are illustrated, but should not be limited by the following examples. Those skilled in this art will also appreciate that all other examples obtained based on this invention without creative work shall fall within the scope of protection of this invention.

Stated in more detailed process terms, some of the more preferred embodiments of the invention may be illustrated, but should not be limited by the following additional exemplary versions of the herein disclosed processes. The process of this invention can also be implemented in other ways different from those described herein. Those skilled in this art will also make similar promotion without violating the connotation of the present invention.

“An embodiment” or “an example” herein means that a particular feature, structure or property may be contained in at least one implementation of the present invention. “In one embodiment” appearing in different places in this specification does not mean the same embodiment, nor an embodiment that is exclusive or selective from other embodiments.

The following examples serve to illustrate the process of the invention to evaluate the similarity of dietary fat to human milk fat. The data of the lipid composition of 309 human milk samples in Wuxi, China, with the 95% confidence interval, is used as the evaluation basis. In the calculation of 95% confidence interval of human milk fat index, some indexes fail to show the normal distribution and some have negative boundary value. However, all the index data on human milk fat, biological samples with a large enough sample size, should follow the normal distribution. Considering the continuity of similarity evaluation, the calculation method of confidence interval under normal distribution is still selected in the examples, and the boundary value of the confidence interval is set to 0 when it is negative.

Based on the existing analysis methods, similarity evaluation is conducted mainly based on the composition of major fatty acids, trace fatty acids, major sn-2 fatty acids, trace sn-2 fatty acids, major triacylglycerols and trace triacylglycerols in below examples.

The contents of fatty acids, sn-2 fatty acids and triacylglycerols of human milk fat (95% confidence interval) are shown in Table 1˜3.

TABLE 1
The content of major and trace fatty acids
of human milk fat (95% confidence interval)

| Major fatty | | Trace fatty | | Trace fatty | |
|---|---|---|---|---|---|
| acids | Range | acids | Range | acids | Range |
| 10:0 | 0.06~2.21 | 6:0 | 0~0.10 | 18:3(n-6) | 0~0.23 |
| 12:0 | 1.19~8.51 | 8:0 | 0.03~0.33 | 20:2(n-6) | 0.12~1.63 |
| 14:0 | 2.20~7.93 | 20:0 | 0.05~0.40 | 20:3(n-6) | 0.22~0.97 |
| 16:0 | 15.07~22.62 | 22:0 | 0~0.27 | 20:4(n-6) | 0.37~1.31 |
| 16:1(n-7) | 0.98~2.39 | 24:0 | 0~0.21 | 20:5(n-3) | 0~0.30 |
| 18:0 | 3.67~8.32 | 14:1(n-5) | 0.02~0.34 | 22:2(n-6) | 0~0.46 |
| 18:1(n-9) | 26.04~37.27 | 20:1(n-9) | 0.22~1.15 | 22:4(n-6) | 0~0.50 |
| 18:2(n-6) | 14.19~25.52 | 22:1(n-9) | 0~0.40 | 22:5(n-3) | 0~0.66 |
| 18:3(n-3) | 0.68~1.73 | 24:1(n-9) | 0~0.34 | 22:5(n-6) | 0.02~0.4 |
| | | | | 22:6(n-3) | 0.22~0.95 |

TABLE 2
The content of major and trace sn-2 fatty acids
of human milk fat (95% confidence interval)

| Major sn-2 | | Trace sn-2 | | Trace sn-2 | |
|---|---|---|---|---|---|
| fatty acids | Range | fatty acids | Range | fatty acids | Range |
| 12:0 | 0.65~10.24 | 6:0 | 0~0.09 | 18:3(n-3) | 0.26~1.23 |
| 14:0 | 4.46~15.52 | 8:0 | 0~0.38 | 18:3(n-6) | 0~0.14 |
| 16:0 | 43.31~58.37 | 10:0 | 0.10~1.36 | 20:2(n-6) | 0.13~0.74 |
| 16:1(n-7) | 1.37~2.74 | 20:0 | 0.12~0.31 | 20:3(n-6) | 0.12~0.66 |
| 18:0 | 1.21~2.56 | 22:0 | 0~0.28 | 20:4(n-6) | 0.43~1.22 |
| 18:1(n-9) | 6.98~17.23 | 24:0 | 0~0.15 | 20:5(n-3) | 0~0.23 |
| 18:2(n-6) | 3.31~13.42 | 14:1(n-5) | 0.02~0.27 | 22:2(n-6) | 0.02~0.59 |
| | | 20:1(n-9) | 0.24~0.70 | 22:4(n-6) | 0~0.60 |
| | | 22:1(n-9) | 0~0.26 | 22:5(n-3) | 0.02~0.70 |
| | | 24:1(n-9) | 0~0.60 | 22:5(n-6) | 0.04~0.51 |
| | | | | C22:6(n-3) | 0.59~1.47 |

TABLE 3
The content of major and trace triacylglycerol
of human milk fat (95% confidence interval)

| Major | | Major | | Trace | |
|---|---|---|---|---|---|
| triacylglycerols | Range | triacylglycerols | Range | triacylglycerols | Range |
| LaLaO | 0~2.21 | POLa | 4.35~10.78 | CaLaLa | 0~0.92 |
| CaPL | 0~4.88 | POL | 17.85~33.02 | BuOP | 0~0.51 |
| LLL | 0~3.49 | PPL | 1.57~7.25 | MLaCa | 0~0.47 |
| LaOL | 0.30~7.20 | MPO | 0.36~3.17 | CaLaL | 0~0.36 |
| CaPO | 0~8.52 | OOO | 0.52~3.73 | CaLaO | 0~1.60 |
| OLL | 2.90~10.35 | POO | 6.81~24.86 | LaLaM | 0~1.15 |
| PLL | 0~11.39 | PPO | 1.56~8.07 | SOO | 0~1.45 |
| MOL | 0~13.58 | POS | 0~2.83 | PPS | 0.04~1.16 |
| LaOO | 0.21~3.42 | | | | |

### Example 1

The similarity coefficients of 4 human milk samples, referred to as HM1, HM2, HIM3 and HMV4, are evaluated with the use of the above evaluation model. The results of fatty acid, sn-2 fatty acid and triacylglycerol composition are shown in Table 4˜6.

TABLE 4
Fatty acids composition of human milk samples (n = 4)

| Fatty acids | HM1 | HM2 | HM3 | HM4 |
|---|---|---|---|---|
| 6:0 | 0.01 | 0.03 | 0.04 | 0.04 |
| 8:0 | 0.17 | 0.14 | 0.14 | 0.13 |
| 10:0 | 1.15 | 1.39 | 1.39 | 0.76 |
| 12:0 | 3.85 | 5.44 | 5.67 | 2.34 |
| 14:0 | 2.85 | 4.58 | 4.79 | 2.48 |
| 14:1(n-5) | 0.02 | 0.03 | 0.03 | 0.05 |
| 16:0 | 17.04 | 19.52 | 19.59 | 22.28 |
| 16:1(n-7) | 1.64 | 1.71 | 1.72 | 2.32 |
| 18:0 | 4.93 | 5.53 | 5.60 | 6.85 |
| 18:1(n-9) | 30.26 | 31.47 | 31.04 | 35.60 |
| 18:2(n-6) | 30.12 | 21.81 | 21.72 | 18.40 |
| 18:3(n-6) | 0.18 | 0.20 | 0.20 | 0.26 |
| 18:3(n-3) | 0.97 | 1.68 | 1.68 | 1.15 |
| 20:0 | 0.15 | 0.18 | 0.17 | 0.15 |
| 20:1(n-9) | 0.39 | 0.47 | 0.48 | 0.60 |
| 20:2(n-6) | 0.56 | 0.51 | 0.51 | 0.40 |
| 20:3(n-6) | 0.59 | 0.47 | 0.48 | 0.39 |
| 20:4(n-6) | 0.86 | 0.64 | 0.65 | 0.61 |
| 20:3(n-3) | 0.07 | 0.08 | 0.08 | 0.07 |
| 22:0 | 0.08 | 0.06 | 0.06 | 0.03 |
| 22:1(n-9) | 0.12 | 0.18 | 0.19 | 0.26 |
| 20:5(n-3) | 0.09 | 0.04 | 0.04 | 0.15 |
| 22:2(n-6) | 0.07 | 0.07 | 0.07 | 0.03 |
| 22:4(n-6) | 0.27 | 0.20 | 0.20 | 0.15 |
| 22:5(n-6) | 0.17 | 0.10 | 0.10 | 0.14 |
| 24:1(n-9) | 0.03 | 0.05 | 0.05 | 0.02 |
| 22:5(n-3) | 0.22 | 0.12 | 0.12 | 0.22 |
| 22:6(n-3) | 0.58 | 0.34 | 0.34 | 0.81 |

TABLE 5
Sn-2 fatty acids composition of human milk samples (n = 4)

| Fatty acids | HMF1 | HMF2 | HMF3 | HMF4 |
|---|---|---|---|---|
| 10:0 | 0.85 | 0.87 | 0.43 | 0.55 |
| 12:0 | 7.14 | 7.29 | 3.16 | 6.60 |
| 14:0 | 8.93 | 9.04 | 4.92 | 10.70 |
| 14:1(n-5) | 0.03 | 0.02 | 0.02 | 0.01 |
| 16:0 | 47.31 | 46.18 | 53.33 | 46.79 |
| 16:1(n-7) | 2.28 | 2.27 | 2.73 | 2.31 |
| 18:0 | 1.72 | 1.71 | 2.34 | 2.64 |
| 18:1(n-9) | 11.80 | 12.04 | 13.81 | 9.58 |
| 18:2(n-6) | 13.69 | 14.14 | 12.10 | 13.58 |
| 18:3(n-6) | 0.12 | 0.13 | 0.17 | 0.06 |
| 18:3(n-3) | 1.02 | 1.16 | 0.96 | 1.19 |
| 20:0 | 0.19 | 0.19 | 0.14 | 0.12 |
| 20:1(n-9) | 0.29 | 0.29 | 0.28 | 0.25 |
| 20:2(n-6) | 0.30 | 0.32 | 0.20 | 0.15 |
| 20:3(n-6) | 0.24 | 0.24 | 0.16 | 0.12 |
| 20:4(n-6) | 0.55 | 0.56 | 0.43 | 0.48 |
| 22:0 | 0.06 | 0.06 | 0.03 | 0.04 |
| 22:1(n-9) | 0.14 | 0.14 | 0.05 | 0.06 |
| 20:5(n-3) | 0.01 | 0.01 | 0.06 | 0.03 |
| 22:2(n-6) | 0.15 | 0.16 | 0.06 | 0.06 |
| 22:4(n-6) | 0.36 | 0.38 | 0.28 | 0.22 |
| 22:5(n-6) | 0.19 | 0.20 | 0.24 | 0.12 |
| 22:5(n-3) | 0.26 | 0.28 | 0.45 | 0.34 |
| 22:6(n-3) | 0.57 | 0.61 | 1.29 | 0.99 |

TABLE 6
Triacylglycerols composition of human milk samples (n = 4)

| Triacylglycerols | HM1 | HM2 | HM3 | HM4 |
|---|---|---|---|---|
| CaLaLa | 0.2 | 0.26 | 0.17 | 0.07 |
| BuPO | 0.31 | 0.11 | 0.07 | 0.36 |
| CaLaM | 0.22 | 0.18 | 0.15 | 0.23 |
| CaLaL | 0.12 | 0.14 | 0.11 | 0.08 |
| LaCaO | 1.28 | 0.58 | 0.58 | 0.42 |
| LaLaM | 0.45 | 0.99 | 0.57 | 0.34 |
| LaLaO | 1.82 | 0.26 | 0.9 | 0.59 |
| CaPL | 5.03 | 2.47 | 1.54 | 2.02 |
| LLL | 1.96 | 1.06 | 1.94 | 1.57 |
| LaOL | 5.45 | 2.18 | 4.59 | 2.42 |
| CaPO | 4.19 | 4.63 | 4.62 | 3.07 |
| OLL | 6.48 | 8.49 | 8.57 | 6.34 |
| PLL | 3.75 | 2.03 | 5.46 | 7.43 |
| MOL | 5.42 | 7.93 | 5.06 | 3.9 |
| LaOO | 1.63 | 2.19 | 1.36 | 0.85 |
| LaPO | 8.2 | 6.67 | 6.46 | 7.33 |
| OPL | 23.27 | 29.36 | 24.5 | 25.63 |
| PPL | 3.25 | 4.62 | 2.09 | 3.35 |
| MPO | 1.43 | 1.77 | 1.55 | 1.93 |
| OOO | 1.46 | 1.85 | 2.46 | 1.94 |
| OPO | 16.85 | 15.92 | 18.46 | 20.8 |
| PPO | 4.13 | 3.57 | 4.24 | 3.43 |
| SOO | 0.14 | 0.23 | 0.28 | 0.33 |
| POS | 1.46 | 1.26 | 1.3 | 2.14 |
| PPS | 0.86 | 0.56 | 0.78 | 0.97 |

The similarity coefficients of 4 human milk samples evaluated with the use of the above evaluation model are shown in Table 7.

TABLE 7
Similarity coefficients of human milk samples (n = 4)

| | G_{III} | | | | | | G_{II} | | | G_{I} |
|---|---|---|---|---|---|---|---|---|---|---|
| | G_{MAFA} | G_{MIFA} | G_{sn-2MAFA} | G_{sn-2MIFA} | G_{MATAG} | G_{MITAG} | G_{FA} | G_{sn-2FA} | G_{TAG} | G |
| HM1 | 98.87 | 100.00 | 99.71 | 99.84 | 99.82 | 100.00 | 99.43 | 99.78 | 99.91 | 99.71 |
| HM2 | 100.00 | 100.00 | 99.23 | 100.00 | 100.00 | 100.00 | 100.00 | 99.62 | 100.00 | 99.87 |
| HM3 | 100.00 | 100.00 | 100.00 | 98.84 | 100.00 | 100.00 | 100.00 | 99.42 | 100.00 | 99.81 |
| HM4 | 100.00 | 99.36 | 99.39 | 97.62 | 100.00 | 100.00 | 99.68 | 98.50 | 100.00 | 99.39 |

The results indicate that the similarity coefficients of 4 human milk samples in each grade indexes are very high, which reflect the accuracy of evaluation model of this patent to some extent.

### Example 2

The similarity coefficients of 2 kinds of human milk fat substrates that have been used in infant formulas as reported, referred to as IF-A (19% of total palmitic acid located at sn-2 position) and IF-B (44.5% of total palmitic acid located at sn-2 position) respectively, are evaluated with the use of the above evaluation model. Clinical trials have shown that IF-B could reduce the loss of total fatty acids and palmitic acid in the faeces of infants compared with IF-A.

The results of total fatty acids and sn-2 fatty acids composition of 2 kinds of human milk fat substrates are given in Table 8˜9 below.

TABLE 8
Fatty acids composition of 2 kinds of human milk fat substrates

| Fatty acids | IF-A | IF-B |
|---|---|---|
| 4:0 | 0.16 | 0.05 |
| 6:0 | 0.24 | 0.1 |
| 8:0 | 0.75 | 0.56 |
| 10:0 | 1.14 | 0.74 |
| 12:0 | 5.57 | 7.74 |
| 13:0 | 0.02 | 0.01 |
| 14:0 | 5.11 | 4.39 |
| 14:1(n-5) | 0.27 | 0.09 |
| 15:0 | 0.35 | 0.16 |
| 15:1 | 0.09 | 0.04 |
| 16:0 | 23.86 | 23.87 |
| 16:1(n-9) | 0.13 | 0.11 |
| 16:1(n-7) | 0.78 | 0.45 |
| 17:0 | 0.3 | 0.17 |
| 17:1 | 0.14 | 0.07 |
| 18:0 | 6.72 | 4.55 |
| 18:1(n-9) | 38.09 | 40.4 |
| 18:2(n-6) | 13.55 | 13.88 |
| 20:0 | 0.38 | 0.26 |
| 18:3(n-3) | 1.07 | 1.2 |
| 20:1(n-9) | 0.45 | 0.45 |
| 20:2(n-6) | 0.06 | 0.06 |
| 20:3(n-6) | 0.04 | 0.03 |
| 20:4(n-6) | 0.2 | 0.19 |
| 22:0 | 0.25 | 0.15 |
| 24:0 | 0.11 | 0.08 |
| 22:4(n-6) | 0.02 | 0.02 |
| 22:5(n-6) | 0.03 | 0.03 |
| 22:5(n-3) | 0.04 | 0.03 |
| 22:6(n-3) | 0.11 | 0.12 |

TABLE 9
Sn-2 fatty acids composition of 2
kinds of human milk fat substrates

| Fatty acids | IF-A | IF-B |
|---|---|---|
| 12:0 | 8.36 | 10.87 |
| 14:0 | 6.39 | 5.65 |
| 16:0 | 13.59 | 31.88 |
| 18:0 | 2.06 | 2.02 |
| 18:1(n-9) | 52.08 | 32.64 |
| 18:2(n-6) | 16.15 | 15.35 |

The similarity coefficients of 2 kinds of human milk fat substrates evaluated with the use of the above evaluation model are given in Table 10 below.

TABLE 10
Similarity coefficients of 2 kinds of human milk fat substrates

| | G_{III} | | | G_{II} | | G_{I} |
|---|---|---|---|---|---|---|
| | G_{MAFA} | G_{MIFA} | G_{sn-2MAFA} | G_{FA} | G_{sn-2FA} | G |
| IF-A | 96.38 | 73.95 | 44.10 | 85.16 | 44.10 | 64.63 |
| IF-B | 92.20 | 84.20 | 66.24 | 88.20 | 66.24 | 77.22 |

The results indicate that the similarity coefficient of IF-B is higher than IF-A, which is consistent with the clinical trial, reflecting the accuracy of evaluation model of this patent to some extent.

### Example 3

The similarity coefficients of 5 infant formulas fat samples, referred to as IF 1˜5, are evaluated with the use of the above evaluation model.

The composition of total fatty acids, sn-2 fatty acids and triacylglycerols of 5 infant formulas fat samples are given in Table 11˜13 below.

TABLE 11
Fatty acids composition of 5 infant formulas fat samples

| Fatty acids | IF1 | IF2 | IF3 | IF4 | IF5 |
|---|---|---|---|---|---|
| 4:0 | 1.09 | 1.91 | 0 | 2.47 | 0.11 |
| 6:0 | 1.02 | 1.4 | 0.03 | 1.72 | 0.65 |
| 8:0 | 1.04 | 1.29 | 0.84 | 1.06 | 6.41 |
| 10:0 | 2.87 | 2.21 | 0.85 | 2.17 | 4.32 |
| 12:0 | 3.83 | 5.18 | 7.12 | 2.42 | 22.97 |
| 14:0 | 7.91 | 7.59 | 3.3 | 7.98 | 6.48 |
| 14:1(n-5) | 0.33 | 0.74 | 0 | 0.94 | 0.08 |
| 16:0 | 27.94 | 20.33 | 7.56 | 24.86 | 7.01 |
| 16:1(n-7) | 0.55 | 0.76 | 0.15 | 0.9 | 0.15 |
| 18:0 | 8.02 | 6.21 | 3.07 | 8.05 | 2.34 |
| 18:1(n-9) | 24.84 | 27.05 | 50.97 | 23.69 | 30.58 |
| 18:2(n-6) | 14.37 | 18.71 | 19.5 | 17.17 | 15.42 |
| 18:3(n-6) | 0.03 | 0 | 0.04 | 0.04 | 0 |
| 18:3(n-3) | 1.36 | 1.97 | 2.08 | 1.7 | 1.82 |
| 20:0 | 0.21 | 0.13 | 0.36 | 0.21 | 0.12 |
| 20:1(n-9) | 0.06 | 0.1 | 0.44 | 0.13 | 0.12 |
| 20:2(n-6) | 0.02 | 0.05 | 0.03 | 0.06 | 0 |
| 20:3(n-6) | 0.07 | 0 | 0.05 | 0.09 | 0 |
| 20:4(n-6) | 0.23 | 0.08 | 0.53 | 0.36 | 0.05 |
| 22:0 | 0.12 | 0.13 | 0.54 | 0.13 | 0.21 |
| 22:1(n-9) | 0.03 | 0.02 | 0.09 | 0 | 0 |
| 20:5(n-3) | 0.05 | 0.04 | 0.02 | 0.05 | 0 |
| 24:0 | 0.11 | 0.05 | 0.19 | 0.08 | 0.09 |
| 22:5(n-6) | 0.04 | 0 | 0.09 | 0.01 | 0 |
| 24:1(n-9) | 0 | 0.02 | 0.04 | 0 | 0 |
| 22:5(n-3) | 0.05 | 0.06 | 0.02 | 0.04 | 0 |
| 22:6(n-3) | 0.07 | 0.04 | 0.25 | 0.14 | 0.07 |

TABLE 12
Sn-2 fatty acids composition of 5 infant formulas fat samples

| Fatty acids | IF1 | IF2 | IF3 | IF4 | IF5 |
|---|---|---|---|---|---|
| 8:0 | 0.5 | 0.41 | 0.16 | 0.62 | 0.31 |
| 10:0 | 2.73 | 1.68 | 0.59 | 2.1 | 1.2 |
| 12:0 | 5.68 | 6.96 | 16.68 | 3.14 | 31.75 |
| 14:0 | 12.26 | 11.26 | 2.72 | 13.05 | 4.29 |
| 14:1(n-5) | 0 | 0.8 | 0.02 | 1.14 | 0.06 |
| 16:0 | 25.68 | 20.59 | 3.24 | 24.6 | 1.39 |
| 16:1(n-7) | 1.21 | 1.02 | 0.12 | 1.33 | 0.11 |
| 18:0 | 4.66 | 2.85 | 1.51 | 4.06 | 0.32 |
| 18:1(n-9) | 23.1 | 24.74 | 44.93 | 21.52 | 34.23 |
| 18:2(n-6) | 17.65 | 23.68 | 24.81 | 23.34 | 23.38 |
| 18:3(n-6) | 0 | 0 | 0 | 0.04 | 0 |
| 18:3(n-3) | 1.36 | 2.62 | 3.65 | 1.68 | 2.22 |
| 20:4(n-6) | 0.16 | 0.05 | 0.18 | 0.2 | 0 |
| 22:6(n-3) | 0.08 | 0.05 | 0.13 | 0.09 | 0.09 |

TABLE 13
Triacylglycerols composition of 5 infant formulas fat samples

| Triacylglycerols | IF1 | IF2 | IF3 | IF4 | IF5 |
|---|---|---|---|---|---|
| BuOM | 1.148 | 1.574 | 0 | 1.269 | 0 |
| CaLaLa | 0.541 | 0 | 1.745 | 0 | 2.79 |
| CoMM + BuMP | 1.281 | 1.192 | 0 | 1.819 | 0 |
| BuOO | 3.104 | 1.145 | 0 | 0.774 | 0 |
| CoOM | 4.482 | 0 | 0 | 0 | 0 |
| BuOP | 0.69 | 2.345 | 0 | 4.128 | 0 |
| LaLaLa | 0 | 0 | 1.837 | 0 | 4.273 |
| CaCaP | 1.148 | 0 | 0 | 0 | 0 |
| CaLaM | 0 | 0.567 | 0 | 1.595 | 0 |
| BuPP | 0.605 | 3.166 | 0 | 5.64 | 0 |
| CoOP | 3.706 | 2.533 | 0 | 2.628 | 0 |
| BuOS | 1.203 | 3.04 | 0 | 8.618 | 0 |
| LaLaM | 0 | 0 | 1.379 | 0 | 2.79 |
| CaLaP | 2.898 | 0 | 0.995 | 0 | 2.478 |
| CoPP | 3.65 | 4.532 | 0 | 8.638 | 0 |
| BuSP | 1.024 | 0 | 0 | 0 | 0 |
| LLLn | 0 | 2.454 | 1.439 | 3.023 | 3.243 |
| CyOO | 0.742 | 0 | 0 | 0 | 0 |
| LaLaO | 3.099 | 0 | 0.078 | 0 | 0.996 |
| CaOM + CyOP | 4.738 | 2.433 | 0 | 5.46 | 0 |
| MMLa | 0 | 0.74 | 0.072 | 2.303 | 0.412 |
| CoPS | 1.124 | 0 | 0 | 0 | 0 |
| LLL | 4.542 | 5.355 | 2.491 | 3.28 | 4.454 |
| LLnO | 2.365 | 3.821 | 1.677 | 2.936 | 2.212 |
| LLnP | 0 | 0.552 | 0 | 1.25 | 0.183 |
| CaOO | 0.604 | 0 | 0 | 0 | 0 |
| LaOM | 1.277 | 0 | 0 | 0 | 0 |
| CaOP | 1.709 | 0 | 0 | 0 | 0.102 |
| LaMP | 0 | 0 | 0.24 | 0 | 0.478 |
| OLL | 4.862 | 10.573 | 7.721 | 3.054 | 4.896 |
| PLL | 4.542 | 7.136 | 3.604 | 3.963 | 3.54 |
| LaOP | 1.684 | 1.339 | 0.042 | 2.607 | 0.121 |
| MMP + PPLa | 1.533 | 1.172 | 0.068 | 1.745 | 0.21 |
| OOL | 3.171 | 9.501 | 10.043 | 1.946 | 3.783 |
| POL | 0 | 0 | 3.049 | 0 | 2.916 |
| MOO | 6.652 | 6.701 | 0 | 5.199 | 0 |
| PPL | 0 | 0 | 0.239 | 0 | 0.303 |
| MPO | 4.771 | 2.712 | 0 | 4.644 | 0 |
| MPP | 1.129 | 1.011 | 0.033 | 1.682 | 0.04 |
| OOO | 2.309 | 13.001 | 55.698 | 1.206 | 52.495 |
| OOP | 8.154 | 4.096 | 4.367 | 4.509 | 3.798 |
| POP | 9.677 | 2.19 | 0.151 | 5.153 | 0.266 |
| PPP | 0.766 | 0.5 | 0.053 | 1.412 | 0 |
| SOO | 0.538 | 0.634 | 1.648 | 0.509 | 1.372 |
| POS | 1.295 | 0.494 | 0.042 | 1.307 | 0.066 |
| PPS | 0.345 | 0.256 | 0 | 0.554 | 0 |

The similarity coefficients of 5 infant formulas fat samples are evaluated with the use of the above evaluation model. The results are shown in Table 14 below.

TABLE 14
Similarity coefficients of 5 infant formulas fat samples

| | G_{III} | | | | | | G_{II} | | | G_{I} |
|---|---|---|---|---|---|---|---|---|---|---|
| | G_{MAFA} | G_{MIFA} | G_{sn-2MAFA} | G_{sn-2MIFA} | G_{MATAG} | G_{MITAG} | G_{FA} | G_{sn-2FA} | G_{TAG} | G |
| IF1 | 88.68 | 22.87 | 71.43 | 47.99 | 64.58 | 95.59 | 55.78 | 59.71 | 80.09 | 65.19 |
| IF2 | 95.96 | −14.82 | 70.09 | 41.87 | 52.16 | 52.44 | 40.57 | 55.98 | 52.30 | 49.62 |
| IF3 | 76.90 | 73.33 | 24.10 | 50.82 | −22.72 | 72.09 | 75.11 | 37.46 | 24.69 | 45.75 |
| IF4 | 96.92 | −18.78 | 70.92 | 37.40 | 69.39 | −18.60 | 39.07 | 54.16 | 25.40 | 39.54 |
| IF5 | 50.55 | −52.24 | 7.28 | 54.04 | −19.04 | 44.27 | −0.84 | 30.66 | 12.61 | 14.14 |

### Example 4

The structured lipids for human milk fat substrates are produced from extra virgin olive oil, tripalmitin, arachidonic acid and docosahexaenoic acid using Novozym 435 and/or Lipozyme TL IM through one-step or two-steps process. At last four kinds of structured lipids, referred to as SL1˜4, are obtained.

The composition of total fatty acids, sn-2 fatty acids and triacylglycerols of 4 kinds of structured lipids are given in Table 15˜17 below.

TABLE 15
Fatty acids composition of 4 kinds of structured lipids

| Fatty acids | SL1 | SL2 | SL3 | SL4 |
|---|---|---|---|---|
| 8:0 | 0 | 0 | 0.5 | 0.6 |
| 10:0 | 1.04 | 1.02 | 1.11 | 0.7 |
| 12:0 | 0.21 | 0.18 | 0.44 | 0.42 |
| 14:0 | 1.97 | 2.11 | 2.47 | 2.54 |
| 16:0 | 36.69 | 44.23 | 35.23 | 40.07 |
| 16:1(n-7) | 1.03 | 0.87 | 0.85 | 0.84 |
| 18:0 | 2.82 | 2.58 | 3.19 | 3.02 |
| 18:1(n-9) | 43.22 | 38.64 | 38.08 | 37.1 |
| 18:2(n-6) | 6.34 | 5.29 | 5.79 | 4.09 |
| 20:0 | 0.29 | 0.23 | 0.33 | 0.28 |
| 18:3(n-6) | 0.08 | 0.06 | 0.46 | 0.45 |
| 18:3(n-3) | 0.47 | 0.39 | 0.33 | 0.3 |
| 22:0 | 0.16 | 0.12 | 0.28 | 0.27 |
| 20:3(n-3) | 0.16 | 0.11 | 0.55 | 0.54 |
| 20:4(n-6) | 3.67 | 2.97 | 6.23 | 5.95 |
| 22:6(n-3) | 1.53 | 1.39 | 3.71 | 2.6 |

TABLE 16
Sn-2 fatty acids composition of 4 kinds of structured lipids

| Fatty acids | SL1 | SL2 | SL3 | SL4 |
|---|---|---|---|---|
| 14:0 | 0 | 1.12 | 1.44 | 1.24 |
| 16:0 | 52.67 | 56.25 | 50.33 | 55.34 |
| 16:1(n-7) | 0 | 0 | 0.87 | 0.73 |
| 18:0 | 0 | 0 | 2.34 | 1.97 |
| 18:1(n-9) | 39.64 | 34.85 | 34.48 | 33.5 |
| 18:2(n-6) | 6.06 | 6.34 | 4.38 | 3.91 |
| 20:4(n-6) | 2.25 | 1.09 | 4.93 | 4.13 |
| 22:6(n-3) | 0.78 | 0.83 | 2.41 | 2.05 |

TABLE 17
Triacylglycerols composition of 4 kinds of structured lipids

| Triacylglycerols | SL1 | SL2 | SL3 | SL4 |
|---|---|---|---|---|
| OAO | 0.98 | 0.75 | 1.21 | 0.74 |
| APA | 2.56 | 1.78 | 6.11 | 5.24 |
| OPD | 1.4 | 1.59 | 2.36 | 2.14 |
| ODO | 0.33 | 0.36 | 1.2 | 0.98 |
| LOL | 0 | 0 | 2.07 | 1.44 |
| LPL | 1.46 | 1.14 | 0.66 | 0.87 |
| MPL | 0.84 | 0.72 | 2.35 | 0.88 |
| POLn | 2.13 | 1.5 | 6.03 | 4.56 |
| SMM | 0 | 0 | 1.44 | 2.59 |
| OOL | 3.22 | 1.68 | 2.11 | 2.02 |
| POL | 6.28 | 4.68 | 6.08 | 4.34 |
| PLP | 2.53 | 3.42 | 2.32 | 2.37 |
| PPM | 1.12 | 1.11 | 0.88 | 0.67 |
| OOO | 8.32 | 6.12 | 7.64 | 6.83 |
| OPO | 25.17 | 28.84 | 23 | 25.96 |
| PPO | 31.35 | 33.95 | 24.82 | 28.64 |
| PPP | 4.5 | 10.32 | 4.02 | 6.23 |
| OOS | 1.83 | 0.86 | 1.38 | 1.15 |
| POS | 4.31 | 2.05 | 3.8 | 3.65 |
| PPS | 0.82 | 0.68 | 0.78 | 0.82 |

The similarity coefficients of 4 kinds of structured lipids evaluated with the use of the above evaluation model are shown in Table 18.

TABLE 18
Similarity coefficients of 4 kinds of structured lipids

| | G_{III} | | | | | | G_{II} | | | G_{I} |
|---|---|---|---|---|---|---|---|---|---|---|
| | G_{MAFA} | G_{MIFA} | G_{sn-2MAFA} | G_{sn-2MIFA} | G_{MATAG} | G_{MITAG} | G_{FA} | G_{sn-2FA} | G_{TAG} | G |
| SL1 | 68.85 | 55.73 | 24.28 | 48.36 | 39.42 | 96.72 | 62.29 | 36.32 | 68.07 | 55.56 |
| SL2 | 62.84 | 59.31 | 31.84 | 52.38 | 42.67 | 100.00 | 61.08 | 42.11 | 71.34 | 58.17 |
| SL3 | 71.34 | 30.46 | 56.52 | 34.86 | 50.52 | 100.00 | 50.90 | 45.69 | 75.26 | 57.28 |
| SL4 | 66.57 | 36.56 | 55.24 | 39.14 | 47.21 | 100.00 | 51.56 | 47.19 | 73.60 | 57.45 |

The major virtue of this invention is that the evaluation highlights each index without the influence of the content of indexes, and quantifies the differences between dietary fat and human milk fat by the similarity coefficients. Another feature is the ability to grade human milk fat and evaluate the similarity of dietary fat to human milk fat.

It should be stated that the above examples are used only to illustrate the procedures of this invention, but not the limitation to them. Those skilled in this art also will appreciate that while the above examples illustrate the application of this invention, the method is applicable to essentially all substances containing lipids. Slight variations in procedures may well present themselves to those skilled in this art without detracting from the scope and spirit of the invention described herein.

1. A method for evaluating the similarity between dietary fats and human milk fat, comprising:
(1) calculating the drift rate of each index of dietary fat, e.g., comparing the actual values of a certain index in dietary fat with the range of standard values of the corresponding index in human milk fat by use of the equation:
$$C = \frac{\vert b - a \vert}{a}$$
where a represents the standard value of an index, b represents the actual value of the index; and if b is more than aₘₐₓ, a is equivalent to aₘₐₓ; if b is less than aₘᵢₙ, a is equivalent to aₘᵢₙ; C, represents the extent of the determined value of a certain index deviating from the theoretical value range, i.e. shift rate; When b is between aₘᵢₙ and aₘₐₓ, C is defined as 0, namely, shift rate is equivalent to 0.
(2) establishing a similarity evaluation model, calculated using the equation:
$$G = {\sum }_{i = 1}^{n} [ ( 1 - \frac{\vert b - a \vert}{a} ) \times 100 ] \times \frac{{d}_{i}}{{\sum }_{i = 1}^{n} {d}_{i}}$$
where dᵢ represents the appointed weight for i^{th} index; dᵢ/Σᵢ₌₁ⁿdᵢ represents the normalized weight for the i^{th} index after normalization; G represents the similarity coefficient of a certain index of dietary fat; the higher G value, the higher the similarity; and the maximum of G value is 100; conversely, the smaller G value, the lower similarity.

2. The method of claim 1 comprising conducting a hierarchical evaluation of multi-grade indexes of dietary fat and calculating the similarity using the equations:
$${G}_{III} = {\sum }_{i = 1}^{n} [ ( 1 - \frac{\vert b - a \vert}{a} ) \times 100 ] \times \frac{{d}_{i}}{{\sum }_{i = 1}^{n} {d}_{i}}$$
$${G}_{II} = {\sum }_{i = 1}^{n} {e}_{i} {G}_{III}$$
$${G}_{I} = {\sum }_{i = 1}^{n} {f}_{i} {G}_{II}$$
where G_{III} is the similarity coefficient of each grade III index of dietary fat to human milk fat; G_{II} is the similarity coefficient of each grade II index of dietary fat to human milk fat; eᵢ is the appointed weight for the i^{th} grade III index, 0≤eᵢ≤1; G_{I} is the similarity coefficient of all grade II index of dietary fat to human milk fat; fᵢ is the appointed weight for the i^{th} grade II index, 0≤fᵢ≤1.

3. The method of claim 1 wherein the standard value of a certain index, a, is expressed as mean n times of standard deviation, using the equation a=ā±nσ, where ā is the mean, σ is the standard deviation.

4. The method of claim 3 wherein the a is the 95% confidence interval and said n=1.96.

5. The method of claim 2 wherein the grade I index is the lipid composition of human milk fat.

6. The method of claim 2 wherein the grade II index contains fatty acids, sn-2 fatty acids, triacylglycerols and complicated lipids.

7. The method of claim 2 wherein the fatty acids in grade III index contains major fatty acids, trace fatty acids and special fatty acids, and the sn-2 fatty acids in grade III index contain major sn-2 fatty acids, trace sn-2 fatty acids and special sn-2 fatty acids.

8. The method of claim 2 wherein the triacylglycerols in grade III index comprises major triacylglycerols and trace triacylglycerols.

9. The method of claim 2 wherein the complicated lipids in grade III index contains phospholipids, sterols, and glycolipids.

10. The method of claim 7 wherein the major fatty acids are the fatty acids that represents more than 1% of the total fatty acids; major sn-2 fatty acids are the fatty acids that represent more than 1% of the total sn-2 fatty acids; major triacylglycerols are the triacylglycerols that represent more than 1% of the total triacylglycerols; trace fatty acids are defined as the fatty acids with less than 1% of the total fatty acids, except for special fatty acids; trace sn-2 fatty acids are defined as the fatty acids with less than 1% of the total sn-2 fatty acids, except for special fatty acids; trace triacylglycerols are defined as the triacylglycerols with less than 1% of the triacylglycerols; special fatty acids that are different from conventional fatty acids due to their special structure of functional group contain odd-chain fatty acids, branched-chain fatty acids, cyclic fatty acids, alkenyl acid.
